(19) United States
(12) Patent Application Publication
(10) Pub. No.: US 2020/0270563 A1
(43) Pub. Date: Aug. 27, 2020

(54) METHOD FOR COUNTING NUMBER OF MICROORGANISMS

(71) Applicant: JNC CORPORATION, Tokyo (JP)

(72) Inventors: Hajime TERAMURA, Kanagawa (JP); Aoi FUJIWARA, Kanagawa (JP)

(73) Assignee: JNC CORPORATION, Tokyo (JP)

(21) Appl. No.: 16/651,355

(22) PCT Filed: Jun. 4, 2018
(86) PCT No.: PCT/JP2018/021320

§ 371 Date: Mar. 27, 2020

(30) Foreign Application Priority Data
Sep. 29, 2017 (JP) 2017-190522

(51) Int. Cl. C12M 1/34; C12M 1/00; C12Q 1/06

(52) U.S. Cl. CPC C12M 41/36; C12M 23/14; C12M 23/20; C12Q 1/06; C12M 23/22

(57) ABSTRACT

Provided is a method in which visibility of colonies of microorganisms in a medium is improved and quantitative counting of the number of microorganisms can be simply and accurately performed. The method of counting the number of microorganisms includes a step of adding a analyte to a composition containing (a) a polymer compound that can form non-flowable transparent gel without passing through dissolution by heating and without depending on cooling, (b) guar gum and (c) a nutritional ingredient and mixing the resulting mixture, a step of culturing the microorganisms contained in the analyte, and a step of counting the number of colonies of the microorganisms.

### TECHNICAL FIELD

The invention relates to a method for simply counting the number of microorganisms in an analyte.

### BACKGROUND ART

As a method for counting the number of microorganisms, a pour plate method, an agar spread plate method and the like are known (Non-Patent literature No. 1). An agar medium used for culturing the microorganisms in the methods described above is a material prepared by solidifying a medium in which a nutritional ingredient and a selective ingredient are dissolved thereinto together with agar, and needs to be pre-prepared prior to culturing and counting the microorganisms. Moreover, in the agar spread plate method, upon applying an analyte onto a plate medium, the analyte is applied onto the medium while the analyte is completely absorbed into the medium, and therefore the method also has had a problem of needing time for operation.

In recent years, development has been made in various manners on dry simple culture equipment in which pre-preparation of the medium is unnecessary in order to detect and count the microorganisms in a simpler and further efficient manner. In such a culture equipment, the medium is formed by moisture of a liquid analyte added thereto when in use, and can be directly provided for culture.

The present inventors also have so far proposed a method by a medium using a gelling agent such as sodium polyacrylate (application for patent JP 2016-189545, application for patent JP 2016-229753). More specifically, the method includes a method in which operation is simplified by directly adding a fluid analyte as a solvent composing a medium into a gelling agent to emerge colonies by culturing microorganisms in an analyte in the medium, and to visualize the microorganisms. According to the methods described above, simple and accurate counting is made also on an analyte in which the number of existing microorganisms is significantly small or operability and simplicity are improved using a size-reduced culture vessel.

### CITATION LIST

### Non Patent Literature

NPL 1: Manual to Practice Bacteriology, 2nd, p 59, 4.3 Bacteria counting and culture method, edited by The Institute of Medical Science, The University of Tokyo, Maruzen Co., Ltd.

### SUMMARY OF INVENTION

### Technical Problem

However, in the method described above, free water is generated inside gel formed by swelling of a gelling agent such as sodium polyacrylate by moisture, and colonies of microorganisms are wholly diffused in the gel, and therefore quantitative counting becomes difficult in several cases.

In such a situation, an object of the invention is to provide a method in which visibility of colonies of microorganisms in a medium is improved, and quantitative counting of the number of microorganisms can be simply and accurately performed.

### Solution to Problem

The present inventors have diligently continued to conduct study in order to solve the problems as described above. As a result, the present inventors have found that, if guar gum is simultaneously used as an ingredient of a medium containing a gelling agent such as sodium polyacrylate, visibility of colonies of microorganisms in the medium is improved, and quantitativeness in counting the number of microorganisms can be secured, and have completed the invention.

More specifically, the invention includes the items described below.

Item 1. Use of a composition for preparing a medium for counting the number of microorganisms, wherein the composition comprises (a) a polymer compound that can form non-flowable transparent gel without passing through dissolution by heating and without depending on cooling, (b) guar gum and (c) a nutritional ingredient.

Item 2. The use of item 1, wherein (a) the polymer compound can be hydrated in an amount 10 times or more its own weight.

Item 3. The use of item 1 or 2, wherein the polymer compound has acrylic acid as a monomer unit.

Item 4. The use of item 3, wherein the polymer compound is polyacrylic acid and/or a salt thereof.

Item 5. The use of any one of items 1 to 4, wherein the composition further comprises (d) a coloration reagent.

Item 6. Use of culture equipment for counting the number of microorganisms, wherein the culture equipment comprises (a) a polymer compound that can form non-flowable transparent gel without passing through dissolution by heating and without depending on cooling, (b) guar gum, (c) a nutritional ingredient, and a culture vessel.

Item 7. A composition for preparing a medium for counting the number of microorganisms, containing (a) a polymer compound that can form non-flowable transparent gel without passing through dissolution by heating and without depending on cooling, (b) guar gum and (c) a nutritional ingredient.

Item 8. The composition according to item 7, wherein (a) the polymer compound can be hydrated in an amount 10 times or more its own weight.

Item 9. The composition according to item 7 or 8, wherein the polymer compound has acrylic acid as a monomer unit.

Item 10. The composition according to item 9, wherein the polymer compound is polyacrylic acid and/or a salt thereof.

Item 11. The composition according to any one of items 7 to 10, further containing (d) a coloration reagent.

Item 12. Culture equipment for counting the number of microorganisms, including the composition according to any one of items 7 to 11, and a culture vessel.

Item 13. The culture equipment according to item 12, wherein the culture vessel includes an upper member and a lower member having a recess.

Item 14. The culture equipment according to item 13, wherein the upper member has a projection having a shape that can be mutually fitted to the recess of the lower member through the composition.

Item 15. The culture equipment according to item 14, wherein the composition is coated onto at least a part of the projection of the upper member and/or the recess of the lower member.

Item 16. The culture equipment according to any one of items 13 to 15, wherein the upper member and/or the lower member is transparent.

Item 17. A method for counting the number of microorganisms, including: a step of adding an analyte to the composition according to any one of items 7 to 11; a step of culturing microorganisms contained in the analyte; and a step of counting the number of colonies of the microorganisms.

Item 18. The method according to item 17, wherein the number of microorganisms in the analyte is 0.1 CFU/mL or less.

Item 19. The method according to item 17 or 18, wherein a weight of the analyte is 10 to 10,000 times a weight of the polymer compound in the composition.

### Advantageous Effects of Invention

According to the invention, the number of microorganisms in an analyte can be simply and accurately counted. In particular, quantitative detection can be achieved even for the number of microorganisms in a small amount existing in an analyte in a large volume. Moreover, if an aspect using a specific culture vessel is applied, the number of microorganisms can be counted with high operability and accuracy by using the analyte in a small volume.

### BRIEF DESCRIPTION OF DRAWINGS

FIG. 1 is a diagram showing one aspect of culture equipment according to the invention, in which (A) shows an orthographic diagram of the culture equipment in a state in which an upper member and a lower member are not stacked, (B) shows one example of a cross sectional view taken along line A-A′ in (A), (B′) shows another example of the cross sectional view taken along line A-A′ in (A), (C) shows one example of a cross sectional view taken along line B-B′ in (A), and (C′) shows another example of the cross sectional view taken along line B-B′ in (A).

FIG. 2 is a diagram showing one aspect of a culture vessel according to the invention, in which (A) shows an orthographic diagram of the culture vessel in a state in which an upper member and a lower member are not fitted, (B) shows one example of a cross sectional view taken along line A-A′ in (A), (B′) shows another example of the cross sectional view taken along line A-A′ in (A), (C) shows one example of a cross sectional view taken along line B-B′ in (A), and (C′) shows another example of the cross sectional view taken along line B-B′ in (A).

FIG. 3 is a diagram showing one aspect of a culture vessel according to the invention, in which (A) shows an orthographic diagram of the culture vessel in a state in which an upper member is fitted to a lower member, (B) shows one example of a cross sectional view taken along line C-C′ in (A), and (C) shows another example of the cross sectional view taken along line C-C′ in (A).

FIG. 4 is an oblique projection diagram showing a use aspect example of a culture vessel according to the invention.

FIG. 5 is a photograph of colonies in 100 milliliters of a gel-shaped medium in Comparative Example 1.

FIG. 6 is a photograph of colonies in 100 milliliters of a gel-shaped medium in Example 1.

### DESCRIPTION OF EMBODIMENTS

A composition of the invention essentially contains (a) a polymer compound that can form non-flowable transparent gel without passing through dissolution by heating and without depending on cooling, (b) guar gum, and (c) a nutritional ingredient. The composition of the invention is a material for preparing the medium for counting the number of the microorganisms. The preparation is ordinarily performed by adding a liquid analyte containing the microorganisms to be counted as a solvent of gel directly composing the medium.

Here, (a) the polymer compound that can form non-flowable transparent gel without passing through dissolution by heating and without depending on cooling plays a role of a gelling agent composing a solidifying medium.

As (a) the polymer compound, a material that can be hydrated in an amount preferably 10 times or more, further preferably 20 times or more, and still further preferably 30 times or more its own weight is suitable. The gel suitable for preparing the medium can be formed by such hydration.

The gel formed has no flowability, and therefore the number of existing microorganisms can be accurately counted. Moreover, the gel preferably causes no syneresis. If syneresis is caused, although existence of colonies of the microorganisms can be qualitatively detected, the number of existing microorganisms becomes hard to be accurately counted in several cases. Here, “syneresis” means that water hydrated in the gel is separated from the gel. Moreover, an expression “causing no syneresis” specifically means that water separated from the gel after being left to stand at room temperature for 60 minutes is preferably 0.5% or less, further preferably 0.1% or less of an initial amount of hydrate, for example.

Moreover, the gel formed is transparent. Thus, the colonies of the microorganisms can be accurately detected from outside without disassembling the culture equipment. In addition, “transparency” herein means that, when the polymer compound is added to distilled water at a concentration at which the gel formed does not flow, visible light transmittance measured by a spectrophotometer (optical path length: 1 cm) is preferably 70% or more (the visible light transmittance of distilled water is taken as 100%), but not limited thereto.

Moreover, the polymer compound can form the gel without passing through dissolution by heating and without depending on cooling. Therefore, the operation is simplified and growth of target microorganisms is not hindered. In addition, “heating” herein means raising temperature from room temperature, and specifically means raising temperature to a level at which the microorganisms become inviable, for example, to a temperature over 60° C. Moreover, “cooling” herein means decreasing temperature from a level upon dissolving the polymer compound into the liquid analyte. Moreover, “room temperature” herein ordinarily means 1 to 40° C., preferably 1 to 30° C., and further preferably 20 to 30° C.

Specific examples of such a polymer compound preferably include a material having acrylic acid as a monomer unit, and as long as the material has acrylic acid as the monomer unit, the material is not limited to a homopolymer, and may be a copolymer or a crosslinked polymer.

Specifically, at least one selected from polyacrylic acid and/or a salt thereof and a derivative thereof (hereinafter, also described as “polyacrylic acids”) is preferred.

The gel formed by polyacrylic acids has no flowability, and is hard to cause syneresis, and therefore the number of existing microorganisms can be accurately counted.

Moreover, the gel formed is transparent. Thus, the colonies of the microorganisms can be accurately detected from outside without disassembling the culture equipment.

Moreover, polyacrylic acids can form the gel without passing through dissolution by heating and without depending on cooling, and therefore the operation of forming the medium is simple, the growth of the target microorganisms is not hindered. As polyacrylic acids, in view of less expensiveness, easy availability and simplicity of gel formation, sodium polyacrylate is particularly preferred.

As a gelling agent to be used for the medium for microorganisms or the like as a main ingredient, agar, carragheenan and locust bean gum or the like is generally used, but the agents described above require heating upon solidifying the liquid analyte, and therefore are unsuitable for directly solidifying the liquid analyte containing the microorganisms.

Moreover, the gel prepared by solidification using the gelling agent described above is also unsuitable therefor in view of low transparency.

Moreover, polyvinyl alcohol is hard to be homogeneously mixed with the liquid analyte, and also has a problem of easily causing syneresis. Moreover, xanthane gum is also hard to be homogeneously mixed with the liquid analyte to easily form lumps, in which gel solidified also easily becomes opaque.

Carboxymethyl cellulose is unable to solidify the liquid analyte to form flowable gel, and therefore is unsuitable for quantitative detection of the microorganisms.

When sodium polyacrylate is used as (a) the polymer compound, from a viewpoint of solidifying capability, a material having a degree of polymerization of 10,000 or more is preferred, and a material having a degree of polymerization of 22,000 or more is further preferred. Moreover, the material may be crosslinked or need not be crosslinked

A concentration of sodium polyacrylate when in use according to the invention is not particularly limited, but is preferably 0.01 g/100 mL to 10 g/100 mL, and further preferably 0.5 g/100 mL to 5 g/100 mL, for example.

Moreover, when (a) any other polymer compound is used, the concentration when in use only needs to be in the range in which solidified gel is formed as long as the ingredient does not adversely affect advantageous effects of the invention.

Then, (b) the guar gum is water-soluble, and viscosity thereof is increased by containing water, and therefore movement of free water within the gel formed by (a) the polymer compound can be suppressed.

In addition, even when the guar gum is added, transparency of the gel of polyacrylic acid or the like, gelation thereof, growth of microorganisms, or a reaction according to a coloration reagent are not hindered.

A concentration of (b) the guar gum when in use thereof according to the invention is not particularly limited, but is preferably 1/200 to 1 times an amount of (a) the polymer compound in terms of a mass ratio, and further preferably 1/40 to ½ times the amount thereof. For example, when 1 to 2 g/100 mL of sodium polyacrylate is used as a concentration in use thereof as (a) the polymer compound, the concentration of (b) the guar gum when in use is preferably 0.01 g/100 mL to 1.0 g/100 mL, and further preferably 0.05 g/100 mL to 0.5 g/100 mL. A medium preferred for counting is formed without hindering gelation of the medium and without causing an insoluble mass (so-called lumps) of guar gum by adjusting the concentration to such an amount.

Then, (c) the nutritional ingredient is applied for growing the target microorganisms.

The nutritional ingredient is not particularly limited, and specific examples thereof preferably include peptone, an animal meat extract, a yeast extract and a fish meat extract.

In testing on drinking water with regard to detection of the microorganisms in tap water or the like, use of a standard agar medium is recommended, and in testing of pharmaceutical water or dialysis water, use of an R2A agar medium is recommended (The Japanese Pharmacopoeia, 17th Edition, G8 Water in General Information). Therefore, a bouillon medium in which agar is excluded from the agar media described above, or an ingredient equivalent thereto is preferable incorporated thereinto as a component of the composition according to the invention.

Then, the composition of the invention further preferably contains (d) the coloration reagent. The reason is that the colonies of the microorganism, the colonies being produced by culture, are easily detected and counted as colored colonies.

Specific examples of the coloration reagent include a redox indicator including 2,3,5-triphenyltetrazolium chloride (TTC) and tetrazolium violet. The indicator described above can be preferably used when all kinds of microorganisms existing in the analyte are desirably counted. When TTC is used, a concentration when in use is preferably 1 mg/L to 100 mg/L, and further preferably 10 mg/L to 50 mg/L.

Moreover, as the coloration reagent, such a material may be used as a substrate (hereinafter, referred to as “enzyme substrate”) to an enzyme owned only by a specific microorganism species, and a compound that can release a pigment compound by being decomposed. The material described above can be preferably used when the specific microorganisms are desirably counted.

Here, the pigment compound may be any of a compound colored under visible light and a fluorescent-colored compound. Specific examples of a functional group that can be released as the colored compound under visible light include a 5-bromo-4-chloro-3-indoxyl group, and 5-bromo-4-chloro-3-indole released is oxidized and fused into 5,5′-dibromo-4,4′-dichloro-indigo, and colored blue. Specific examples of a functional group that can be released as the fluorescent-colored compound include a 4-methyl umbelliferryl group, and 4-methylumbelliferone released emits fluorescence under irradiation with ultraviolet light.

To take the enzyme substrate as an example, when the target microorganisms are a Coliform group, 5-bromo-4-chloro-3-indoxlyl-beta-D-galactopyranoside (X-GAL) or 5-bromo-4-chloro-3-indoxyl-beta-D-glucuronic acid can be preferably used, in a case of Staphylococcus aurei, 5-bromo-4-chloro-3-indoxyl phosphate (X-phos) can be preferably used, in a case of Enterococcus, 5-bromo-4-chloro-3-indoxlyl-beta-D-glucopyranoside (X-GLUC) can be preferably used, and in a case of fungi, X-phos, 5-bromo-4-chloro-3-indoxyl acetate or 5-bromo-4-chloro-3-indoxyl butyrate can be preferably used, respectively. Further, when all of the microorganism species are desirably detected, all of the substances described above may be combined and used.

A concentration of the enzyme substrate when in use is preferably 0.01 to 1.0 g/L, and further preferably 0.2 to 1.0 g/L.

The composition of the invention may further arbitrarily contain a selective substance, an antibacterial substance, inorganic salts, saccharides, a thickening agent, a pH adjuster or the like, as long as the ingredient does not adversely affect advantageous effects of the invention.

Specific examples of the selective substance include an antibiotic such as polymixin B and vancomycin, and a surfactant such as sodium lauryl sulfate (SDS), Tween 80 and a bile salt such as sodium cholate.

Specific examples of the antibacterial substance include polylysine, protamine sulfate, glycine and sorbic acid.

Specific examples of the inorganic salts include an inorganic acid metal salt such as sodium chloride and sodium thiosulfate, and an organic acid metal salt such as sodium pyruvate, ferric ammonium citrate and sodium citrate.

Specific examples of the saccharides include glucose, lactose, sucrose, xylose, cellobiose and maltose.

Specific examples of the viscosity improver include starch and a derivative thereof, hyaluronic acid, an acrylic acid derivative, polyether and collagen.

Specific examples of the pH adjuster include sodium carbonate, sodium hydrogen-carbonate and citric acid. In addition, from a viewpoint of the growth of the target microorganisms, the composition according to the invention is such a composition to be preferably 6.0 to 8.0, and further preferably 6.5 to 7.5 in the pH when in use.

The composition of the invention may be provided as culture equipment for counting the number of microorganisms in combination with a culture vessel.

The culture vessel contained in such culture equipment is a vessel for directly housing the liquid analyte therein ordinarily without applying treatment such as concentration and dilution thereto, and mixing the liquid analyte with the composition according to the invention therein to cause gelation of the polymer compound contained in the composition to form a medium, and culturing the microorganisms.

A form of the culture vessel is not particularly limited, and may be a vessel as long as a required amount of the fluid analyte can be sufficiently housed. For example, in order to mix the composition of the invention and the liquid analyte by shaking thereof, a vessel that has a cylindrical shape or the like and is of a material that is hard to cause deformation is preferred. Moreover, for example, in order to mix the composition of the invention and the liquid analyte by rubbing and pressing together with the vessel, a vessel of a material that is easily deformed and flexible is preferred. Specific examples of the vessel preferably include a bag-shaped vessel of a polymer such as a polyvinyl base and a polyethylene base, and a vessel with a sealing device such as a lid and a fastener is further preferred. Moreover, the culture vessel is preferably transparent in view of easiness of counting the colonies of the microorganism from outside of the vessel. In addition, transparency herein may be at a degree at which a side opposite to the vessel can be visually observed through the vessel, and more specifically, the visible light transmittance is preferably 70% or more, but not limited thereto. A dose capable of being housed therein is not particularly limited, but specific examples thereof preferably include 100 milliliters to 1,000 milliliters, which is suitable for applying the vessel to a large volume of analyte containing a small amount of microorganisms.

While the analyte has been brought into contact with a preformed medium to culture and count the microorganisms in existing culture equipment, the culture equipment for counting the number of microorganisms according to the invention is different therefrom in that the equipment is preferable for a use aspect in which the polymer compound is gelled by using the liquid analyte itself as a solvent in the culture vessel, and the microorganisms in the analyte are cultured inside the gel and the resulting material is provided for counting the microorganisms.

Moreover, the culture vessel may be formed into a plate (sheet) form, and in such a form, the culture vessel preferably has a small size.

Specific examples thereof include a form of a housing vessel prepared by stacking a general petri dish or a concave dish-shaped sheet and a plate-shaped or convex sheet. The colonies of the microorganisms can be further easily counted by forming the vessel into the plate-shaped form. Moreover, size reduction is suitable for detecting the microorganisms by using a small amount of the analyte, for example, about 1 milliliter of the analyte to facilitate processing of a plurality of analytes in parallel at one time. In the case of such a small size and a plate-shape culture vessel, the culture vessel can also be used for a diluted analyte, and therefore such a vessel becomes preferable also when the number of the microorganisms in the analyte is 300 CFU/mL or less, for example.

One aspect of the plate-shape culture vessel will be described with reference to drawings.

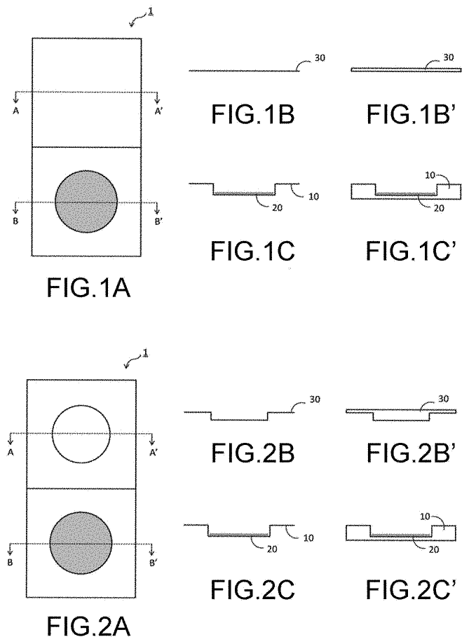

Such a culture vessel includes upper member (30), and lower member (10) having a recess (FIG. 1). The culture equipment is ordinarily used by putting upper member (30) on the recess of lower member (10) in a manner of covering the recess of lower member (10). In a state of putting upper member (30) thereon, a proper space exists between the upper member and the recess of the lower member, and in the aspect in which composition (20) for preparing the medium according to the invention is contained therein, culture equipment (1) of the invention is formed. In such a state, a space surrounded by the upper member, and a bottom surface and a side surface of the recess of the lower member serves as the space in which a medium formed of the composition and an analyte exists (hereinafter, also described as “medium region”).

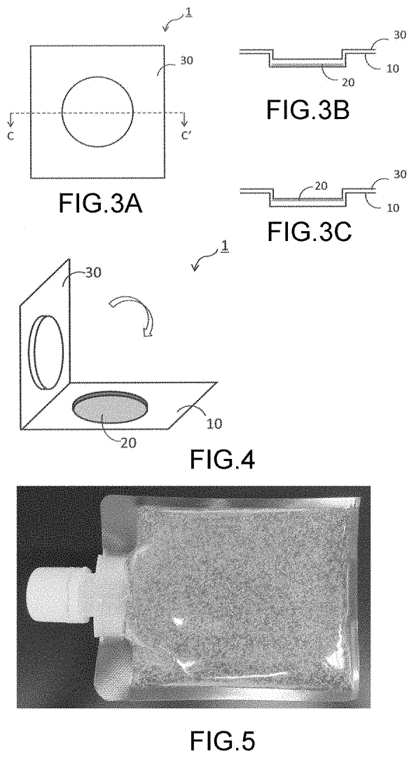

In a preferred aspect, upper member (30) has a projection having a shape that can be mutually fitted to the recess of lower member (10) through composition (20) for preparing a medium (FIG. 2). In the aspect described above, for example, as shown in FIG. 3, a columnar projection of the upper member is fitted to a columnar recess of the lower member, the recess having a diameter somewhat larger than a diameter of the projection. In a fitted state, a top surface of the projection of the upper member and a bottom surface of the recess of the lower member need not be completely brought into close contact with each other, and the proper space exists between the projection of the upper member and the recess of the lower member, and in the aspect in which the composition for preparing the medium according to the invention is contained therein, culture equipment (1) of the invention is formed. In the fitted state, the space surrounded by the top surface of the projection of the upper member, and the bottom surface and the side surface of the recess of the lower member serves as the medium region.

A volume of the medium region can be arbitrarily designed depending on a kind of the analyte to be counted or a scale of examination. A size of the medium vessel is preferably reduced, as a volume of about 1 milliliter, for example. Moreover, the medium region is preferably designed in such a manner that the medium region is not excessively large (depth of the recess of the lower member is not excessively large) in comparison with an amount of the analyte so that the analyte can be spread wholly to the culture region by pressing or the like, upon putting the upper member on the lower member. Moreover, the medium region is preferably designed in such a manner that, when the upper member has the projection, the medium region is not excessively large (height of the projection of the upper member is not excessively small) in comparison with the amount of the analyte so that the projection can spread out the analyte wholly to the medium region by fitting both.

Meanwhile, if the colonies are vertically stacked, the colonies become hard to be accurately observed and counted. Therefore, the medium region is preferably designed in such a manner that the medium region is not excessively small in comparison with the amount of the analyte (bottom area of the recess in a manner of being not excessively large in a thickness of the medium region).

A thickness of the medium region is preferably adjusted to 0.1 to 1.0 millimeter, for example, but not limited thereto.

The projection of the upper member and the recess of the lower member may have an arbitrary shape as long as a fittable shape is applied. Moreover, the top surface of the projection of the upper member and the bottom surface of the recess of the lower member may be either flat or curved, but from a viewpoint of operability, a flat surface is preferred.

The composition for preparing the medium according to the invention is preferably coated onto a portion of the upper member, the portion facing the recess of the lower member upon being put on the lower member, more specifically, at least a part of the portion forming the medium region and/or the recess of the lower member. In an aspect in which the upper member has the projection, the composition for preparing the medium is preferably coated onto at least a part of the projection of the upper member and/or the recess of the lower member. The coated site is ordinarily a portion facing the medium region when the upper member is fitted to the lower member, and is preferably at least a part of the top surface of the projection of the upper member and/or the bottom surface of the recess of the lower member, and is further preferably a whole thereof. In a preferred aspect of the invention, the composition for preparing the medium is coated wholly onto the bottom surface of the recess of the lower member.

According to the present aspect, a material of the upper member and/or the lower member is not particularly limited, and a polyacrylic, polyvinyl-based, polyethylene-based or polyester-based polymer or the like can be adopted. Moreover, rigidity of the material does not matter in particular, but when the upper member has no projection, the rigidity at a moderately deformable level is preferred so as to facilitate pressing after addition of a liquid analyte.

According to the present aspect, the upper member and/or the lower member is preferably transparent, and the upper member and the lower member are further preferably transparent. Thus, the colonies of the microorganisms to be counted can be easily observed and counted from outside without disassembling the culture vessel.

In addition, transparency herein may be at a degree at which a side opposite to the5 member can be visually observed through the member, and more specifically, the visible light transmittance is preferably 70% or more, but not limited thereto.

In the culture vessel according to the present aspect, the upper member and the lower member may be separated, discretely, or may be united.

For example, a part of the upper member and a part of the lower member may be continuous by sharing one side as shown in FIG. 4. When the culture equipment has such an aspect, the culture equipment can be used preferably by fitting the projection of the upper member to the recess of the lower member by folding the culture vessel, stacking the upper member with the lower member and putting the upper member on the lower member.

Moreover, in the culture vessel according to the present aspect, the upper member and the lower member may have a plurality of projections and recesses, respectively. More specifically, the culture equipment may have an aspect in which a plurality of medium regions are formed when in use, which is suitable for treating a plurality of analytes in parallel at one time.

The culture equipment according to the present aspect can be produced by an arbitrary method, and one example will be described.

An acrylic plate or the like having a suitable size is used to be applied as an upper member and a lower member. A projection of the upper member and a recess of the lower member only needs to be prepared by adhesion or hollowing of the acrylic plate, pressing using a mold or the like, molding by injection, or the like.

As the composition for preparing the medium according to the invention, a material prepared by dissolving or suspending the ingredient into a nonaqueous solvent is partially or wholly applied onto the projection of the upper member and/or the recess of the lower member, and then the resulting material is dried. Thus, the composition for preparing the medium according to the invention can be coated onto the culture equipment.

Here, the nonaqueous solvent may be a solvent that can volatilize under ordinary temperature and ordinary pressure, and specific examples thereof preferably include lower alcohol such as ethanol, methanol, propanol and butanol. If the nonaqueous solvent described above is used, the composition for preparing the medium can be coated thereon without gelling (a) the polymer compound during production, and therefore the culture equipment can be easily produced.

The composition for preparing the medium for counting the number of microorganisms and the culture equipment containing the composition according to the invention can be preferably used in the counting method of the invention.

The counting method of the invention includes a step of adding the analyte to the composition of the invention, a step of culturing the microorganisms contained in the analyte and a step of counting the number of the colonies of the microorganisms.

The composition of the invention and the liquid analyte added thereto are ordinarily mixed. Mixing can be performed by an arbitrary method, and for example, the mixing may be performed by shaking or rubbing both together with the vessel or by stiffing with a sterile device.

Culturing conditions of the microorganisms are not particularly limited, and are properly selected according to a kind of the target microorganisms, but are preferably 24 to 48 hours at 35 ±2° C., for example.

The colonies formed by growth of the target microorganisms emerge in the medium after culture, and the colonies can be confirmed by visual observation or the like, and the number of the colonies can be accurately counted.

The counting method of the invention can be preferably used for an analyte in which an amount of existing microorganisms is small, more specifically cleanliness is high. For example, the counting method is preferred when the number of microorganisms in the analyte is 0.1 CFU/mL or less at a level at which the microorganisms are unable to be detected by ordinary inspection using 1 milliliter.

In general, if the analyte in which the amount of existing microorganisms is large, the colonies can be counted by appropriately diluting the analyte so as to be suitable for the detection method, but when the amount of existing microorganisms is small, concentration is complicated or difficult in several cases. Even in such a case, the counting method of the invention is useful in that the number of microorganisms can be simply and accurately detected.

Moreover, the counting method of the invention is also useful even for a large amount of the liquid analyte in view of capability of directly providing the analyte for counting without applying pretreatment thereto. For example, the counting method is preferred when an analyte weight is high in corresponding to a hydration ability of (a) the polymer compound in the composition according to the invention, for example 10 to 10,000 times the weight when the analyte is of sodium polyacrylate. Alternatively, the counting method is preferred when the analyte is in a large amount, for example, in 100 milliliters or more.

Moreover, when the composition of the invention is used for counting the number of microorganisms in the aspect in which the analyte is contained in the plate-shape culture vessel described above, the counting method may be performed as described below.

More specifically, the counting method can be carried out by a step of adding the analyte to the recess of the lower member, a step of putting the upper member on the recess of the lower member, a step of culturing the microorganisms contained in the analyte, and a step of counting the number of colonies of the microorganisms. In the step of putting the upper member on the lower member, the recess is further preferably pressed from outside the equipment. Thus, the analyte added to the recess of the lower member is homogeneously spread out wholly to the medium region. Moreover, (a) the polymer compound in the composition for preparing the medium is further quickly gelled by moisture of the analyte, and the medium is easily formed. Moreover, when the culture equipment in which the upper member has the projection having a shape that can be mutually fitted to the recess of the lower member through the composition for preparing the medium is used, the counting method preferably includes a step of adding the analyte to the recess of the lower member in the culture equipment, a step of fitting the projection of the upper member on the recess of the lower member, a step of culturing the microorganisms contained in the analyte, and a step of counting the number of colonies of the microorganisms.

The analyte added to the recess of the lower member is homogeneously spread out wholly to the medium region by fitting the projection of the upper member to the recess of the lower member. Moreover, (a) the polymer compound in the composition for preparing the medium is further quickly gelled by moisture of the analyte, and the medium is easily formed.

The analyte to which the counting method according to the invention can be applied is not particularly limited, and specific examples thereof preferably include a liquid analyte such as drinking water, soft drink, industrial water, pharmaceutical water, dialysis water and urine. Moreover, the analyte may be a culture solution prepared by preculturing the analyte described above in tryptic soy broth or the like.

### EXAMPLES

Next, the invention will be described in greater detail by way of Examples. The invention is not limited by the Examples.

(1) Preparation of composition for preparing medium for counting microorganisms

A material having a formulation shown in Table 1 was mixed in a transparent and colorless plastic spout bag having a volume of 100 mL to prepare a composition in Comparative Example 1.

Moreover, 0.2 g/100 mL of guar gum was also added to the material having the formulation shown in Table 1 in a similar manner to prepare a composition in Example 1.

Moreover, 0.2 g/100 mL of xanthan gum was also added to the material having the formulation shown in Table 1 in a similar manner to prepare a composition in Comparative Example 2.

TABLE 1

| Material name | Formulation (g) in 100 mL |
|---|---|
| Casein peptone | 0.25 |
| Yeast extract | 0.125 |
| Glucose | 0.05 |
| Sodium polyacrylate | 2 |
| Sodium carbonate | 0.2 |
| TTC | 0.002 |
| | PH 7.2 |

* As sodium polyacrylate, Aqualic CA (Nippon Shokubai Co., Ltd.) was used.

(2) Preparation of Strain Inoculated

As a specimen strain, Escherichia coli NBRC102203 were used. The strain was precultured in a tryptic soy agar medium for 24 hours, and then was suspended into sterile physiological saline using a sterile swab to be a concentration corresponding to McFarland Turbidity Standard No. 1 (about 3.0×10⁸ CFU/mL), and taken as a bacteria stock solution. Then, a bacteria diluted solution having a concentration of several CFU/mL was prepared by repeating 10-fold step dilution of the bacteria stock solution with the sterile physiological saline into a concentration of 10⁸ CFU/mL. Then, a sample solution in which only several CFU of microorganisms existed in 100 mL was prepared by adding 1 mL of the bacteria diluted solution to 99 mL of sterile water. Then, 100 mL of the sample solution was added to a bag in which each of the compositions in Example 1 and Comparative Examples 1 to 2 was stored, and the resulting assembly was rubbed together with the vessel for several minutes and well mixed to solidify the sample. After the sample was gelled, the sample was cultured at 35° C. for 24 hours, and then presence or absence of growth was confirmed.

(3) Results

When the composition in Comparative Example 1 was used, dissolution and also a gelation speed were satisfactory, and gel having transparency was formed, but colonies were diffused wholly into the medium after 24 hours of culture by presence of free water in sodium polyacrylate gel, and therefore counting was unable to be made (see FIG. 5).

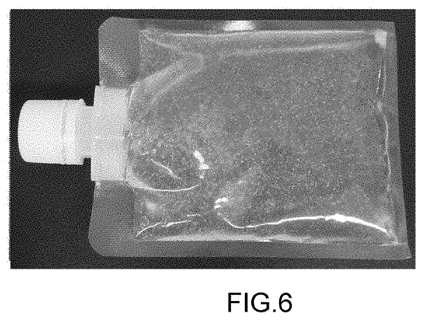

In contrast, when the composition in Example 1 was used, dissolution and also a gelation speed were satisfactory, and gel having transparency was formed, and colonies were not diffused wholly into the medium even after 24 hours of culture because free water in sodium polyacrylate gel was fixed, and therefore accurate counting of colonies was able to be made (see FIG. 6).

Moreover, when the composition in Comparative Example 2 was used, the composition was not completely dissolved therein upon adding a liquid analyte, and lumps were caused into cloudy gel.

Then, colonies were diffused to the whole of the medium after 24 hours of culture, and therefore counting of the colonies was impossible.

TABLE 2

| | State of | | Dissolution |
|---|---|---|---|
| | colonies | Transparency | and gelation |
| Example 1 | No diffusion and | Satisfactory | Satisfactory |
| | countable | transparency | (gelled |
| | | | within 3 |
| | | | minutes) |
| Comparative | Diffused and | Satisfactory | Satisfactory |
| Example 1 | uncountable | transparency | (gelled |
| | | | within 3 |
| | | | minutes) |
| Comparative | Diffused | Cloudy | Not dissolved |
| Example 2 | uncountable | | completely |
| | | | and lumps were |
| | | | caused |

### INDUSTRIAL APPLICABILITY

According to the invention, the number of microorganisms in an analyte can be simply and accurately counted with high visibility, and therefore the art of the invention is useful.

### REFERENCE SIGNS LIST

1 Culture equipment

10 Lower member

20 Composition for preparing medium for counting the number of microorganisms

30 Upper member

1. Use of a composition for preparing a medium for counting the number of microorganisms, wherein the composition comprises (a) a polymer compound that can form non-flowable transparent gel without passing through dissolution by heating and without depending on cooling, (b) guar gum and (c) a nutritional ingredient.

2. The use of claim 1, wherein (a) the polymer compound can be hydrated in an amount 10 times or more its own weight.

3. The use of claim 1, wherein the polymer compound has acrylic acid as a monomer unit.

4. The use of claim 3, wherein the polymer compound is polyacrylic acid and/or a salt thereof

5. The use of claim 1, wherein the composition further comprises (d) a coloration reagent.

6. Use of culture equipment for counting the number of microorganisms, wherein the culture equipment comprises (a) a polymer compound that can form non-flowable transparent gel without passing through dissolution by heating and without depending on cooling, (b) guar gum, (c) a nutritional ingredient, and a culture vessel.

7. A composition for preparing a medium for counting the number of microorganisms, comprising (a) a polymer compound that can form non-flowable transparent gel without passing through dissolution by heating and without depending on cooling, (b) guar gum and (c) a nutritional ingredient.

8. The composition according to claim 7, wherein (a) the polymer compound can be hydrated in an amount 10 times or more its own weight.

9. The composition according to claim 7, wherein the polymer compound has acrylic acid as a monomer unit.

10. The composition according to claim 9, wherein the polymer compound is polyacrylic acid and/or a salt thereof.

11. The composition according to claim 7, further comprising (d) a coloration reagent.

12. Culture equipment for counting the number of microorganisms, comprising the composition according to claim 7, and a culture vessel.

13. The culture equipment according to claim 12, wherein the culture vessel includes an upper member and a lower member having a recess.

14. The culture equipment according to claim 13, wherein the upper member has a projection having a shape that can be mutually fitted to the recess of the lower member through the composition.

15. The culture equipment according to claim 14, wherein the composition is coated onto at least a part of the projection of the upper member and/or the recess of the lower member.

16. The culture equipment according to claim 13, wherein the upper member and/or the lower member is transparent.

17. A method for counting the number of microorganisms, comprising:
a step of adding an analyte to the composition according to claim 7;
a step of culturing microorganisms contained in the analyte; and
a step of counting the number of colonies of the microorganisms.

18. The method according to claim 17, wherein the number of microorganisms in the analyte is 0.1 CFU/mL or less.

19. The method according to claim 17, wherein a weight of the analyte is 10 to 10,000 times a weight of the polymer compound in the composition.
